(19) United States
(12) Patent Application Publication
(10) Pub. No.: US 2020/0268300 A1
(43) Pub. Date: Aug. 27, 2020

(54) SYSTEMS AND METHODS FOR PREDICTING MOOD, EMOTION AND BEHAVIOR OF NON-RECUMBENT SUBJECTS

(71) Applicant: REFAEL SHAMIR, JERUSALEM (IL)

(72) Inventor: REFAEL SHAMIR, JERUSALEM (IL)

(21) Appl. No.: 16/643,909

(22) PCT Filed: Sep. 3, 2018
(86) PCT No.: PCT/IB2018/056713

§ 371 Date: Mar. 3, 2020

Related U.S. Application Data
(60) Provisional application No. 62/553,856, filed on Sep. 3, 2017.

(51) Int. Cl. A61B 5/16; A61B 5/0205; A61B 5/11; A61B 5/00; A47C 1/00; A47C 7/62

(52) U.S. Cl. CPC A61B 5/165; A61B 5/0205; A61B 5/1123; A61B 5/6891; A61B 5/0077; A61B 5/02405; A61B 5/6893; A61B 5/163; A47C 1/00; A47C 7/62; A61B 5/4803

(57) ABSTRACT

Systems and methods for predicting emotional state, behavioral state, or mood of a subject including a non-contacting physiologic sensing apparatus, as well as, possibly, an image capture sensor or a microphone, which may be incorporated into an article of furniture, for detecting parameters indicative of emotional state of a non-recumbent subject.

### FIELD OF THE INVENTION

The disclosure herein relates to systems and methods for predicting emotional state, behavioral state, or mood of a subject. In particular, the disclosure relates to the use of non-contacting physiologic sensing apparatus to detect parameters indicative of emotional state of a subject.

### CROSS-REFERENCE TO RELATED APPLICATIONS

This application claims priority and benefit from U.S. Provisional Patent Application No. 62/553,856, filed Sep. 3, 2017, the contents and disclosure of which are incorporated herein by reference in their entirety.

### BACKGROUND OF THE INVENTION

Emotion and/or mood recognition in a user has been previously proposed. However automatic emotion recognition and/or emotion detection accuracy has been low due to complex algorithms based upon analysis of facial features and/or acoustic cues alone and/or body gesture recognition. In many cases, speech must also be analyzed.

Historically recognition accuracy depends on the number of emotion categories to be recognized, how distinct they are from each other, and the cues employed for emotion recognition and/or emotion detection. Even with complex algorithms, happiness and anger are difficult to distinguish from one another. Although recognition may improve with additional modalities (e.g., facial cues combined with acoustic cues), even with only about four emotional categories to choose from many existing systems achieve 50% recognition accuracy or less.

The need remains, therefore, for efficient and accurate systems for predicting emotional state of a subject. The invention described herein addresses the above-described needs.

### SUMMARY OF THE EMBODIMENTS

It is noted that the emotional state, behavioral state, or mood of a subject may influenced by causal factors and may result in measurable effects. For example, the causal factors influencing emotional state, behavioral state, or mood include environmental stimulation such as social interaction, content consumed, weather, circadian rhythm, time of day, location, awareness of current events and the like.

Measurable effects of emotional state, behavioral state or mood, may include physiological states such as respiration rate, heart rate, shiver response, perspiration rate, blink rate, pupil dilation, head movement rate, eye movement rate, and the like as well as combinations thereof.

One aspect of some embodiments of the invention relates to use of a non-contacting physiologic sensor and/or a wearable sensor to monitor a healthy subject. Optionally, the subject is exposed to content during said measurements. In some embodiments, the sensor measures heart rate data, respiration rate data, body vibration rate data, perspiration rate data, blink rate, pupil dilation, head movement rate data or the like as well as rates of change in those rates.

Alternatively or additionally, in some embodiments, information pertaining to body posture while seated and/or time of day is added to the sensor data. Alternatively or additionally, in some embodiments the content includes game content and/or audio content and/or video content and/or still images and/or questions. In some exemplary embodiments of the invention, physiologic parameter data is collected without a camera and/or without a microphone. The physiologic parameter data may be indicative or representative of the emotional state of the subject.

Another aspect of some embodiments of the invention relates to registration of physiologic parameter data on a common timeline with presented content. In some embodiments, the physiologic parameter data includes heart rate data, respiration rate data, body vibration data, or the like as well as rates of change in those rates. Alternatively or additionally, in some embodiments the content includes game content and/or audio content and/or video content and/or still images and/or questions. In some exemplary embodiments of the invention, physiologic parameter data is collected by a sensor, which does not contact the subject. Alternatively or additionally, in some embodiments physiologic parameter data is collected without a camera and/or without a microphone. Alternatively or additionally, in some embodiments physiologic parameter data is collected without a wearable device.

Another aspect of some embodiments of the invention relates to an article of furniture including at least one seating platform, or “seat”, and a sensor designed and configured to gather physiologic parameter data from a subject seated in said seat without contacting the subject. According to various exemplary embodiments of the invention, the sensor is integrally formed with or attached to the piece of furniture. In some embodiments, the physiologic parameter data includes heart rate data, respiration rate data, body vibration data, or the like as well as rates of change in those rates. Alternatively or additionally, in some embodiments, information pertaining to body posture while seated and/or time of day is added to the sensor data. In some exemplary embodiments of the invention, the sensor includes a data port configured to transmit a time stamped log of the physiologic parameter data to a data processor. According to various exemplary embodiments of the invention the data port is wired (e.g. USB) or wireless (e.g. Bluetooth).

Another aspect of some embodiments of the invention relates to a system including a registration module that impose a common timeline on a physiologic data sensor and a content presentation device. In some exemplary embodiments of the invention, the physiologic data sensor collects data on heart rate data, respiration rate data, body vibration data, or the like as well as rates of change in those rates without contacting the subject. According to various exemplary embodiments of the invention, the content presentation device includes a game console and/or audio playback device and/or video playback device and/or digital picture frame and/or audio recorder.

It will be appreciated that the various aspects described above relate to solution of technical problems associated with emotional response of subjects to perceptible measurement devices (e.g. electrodes that contact the skin).

Alternatively or additionally, it will be appreciated that the various aspects described above relate to solution of technical problems related to simplification of analysis of emotional response.

In some exemplary embodiments of the invention there is provided a method method for monitoring a non-recumbent subject outside a medical institution, such as a hospital, clinic or the like. The method comprises: (a) placing a non-contacting physiologic sensor within operational distance of the subject; and (b) gathering, using said non-contacting physiologic sensor, data pertaining to at least one parameter selected from the group consisting of heart rate, respiration and body-vibrations. The method may further include inferring the emotional state of the subject.

In some embodiments the method includes logging data for each of the at least one parameter on a common timeline. Alternatively or additionally, in some embodiments the method includes logging non-physiologic data on the common timeline. Alternatively or additionally, in some embodiments the method includes using a data processor to calculate a coefficient based on two or more of the parameters. Alternatively or additionally, in some embodiments the method includes using a data processor to calculate a rate of change for one or more of the parameters. Alternatively or additionally, in some embodiments the sensor is positioned in or on or near a seat. Alternatively or additionally, in some embodiments the method includes placing one or more additional non-contacting physiologic sensors within operational distance of the healthy subject. Alternatively or additionally, in some embodiments the method includes activating the sensor only when the subject is within the operational distance.

In some exemplary embodiments of the invention there is provided an article of furniture including: (a) a seating platform; and (b) a non-contacting physiologic sensing apparatus operable to monitor a subject sitting in said seating platform and to gather data pertaining to at least one physiological parameter of said subject. The non-contacting physiologic sensing apparatus may be installed in or on the item of furniture. In some embodiments the sensor is installed at a location selected from the group consisting of within said seating platform, below said seating platform, and on an edge of said seating platform. Alternatively or additionally, in some embodiments the article of furniture includes a back support and the sensor is installed in or on the back support. Alternatively or additionally, in some embodiments the article of furniture includes an armrest, a footrest or a headrest and the sensor is installed in or on the armrest, footrest or headrest. Alternatively or additionally, in some embodiments the article of furniture includes a seat belt, wherein the sensor is installed in or on the seat belt. Alternatively or additionally, in some embodiments the article of furniture includes two or more of the sensors installed at different locations. Alternatively or additionally, in some embodiments the sensor includes reversible attachment hardware. Alternatively or additionally, in some embodiments the article of furniture includes an occupancy detector that activates the sensor only when the seat is occupied.

In some exemplary embodiments of the invention there is provided a system including: (a) a first memory buffer receiving a time stamped output from a non-contacting physiologic sensor monitoring a subject exposed to time stamped content; (b) a second memory buffer containing the time stamped content; and (c) a temporal registration module registering the time stamped output on the time stamped content to produce a log file of physiologic responses to the content. In some embodiments a temporal registration module employs at least one clock selected from the group consisting of a clock embedded in the content, a clock associated with the sensor and a clock providing an output signal to both the sensor and a content presentation device. Alternatively or additionally, in some embodiments the system includes a presentation module that presents the time stamped output from the non-contacting physiologic sensor concurrently with the content. Alternatively or additionally, in some embodiments the presentation module presents the time stamped output from the non-contacting physiologic sensor graphically. Alternatively or additionally, in some embodiments the presentation module presents the time stamped output from the non-contacting physiologic sensor numerically. Alternatively or additionally, in some embodiments the system includes a data processor which calculates a coefficient based on two or more of the parameters. Alternatively or additionally, in some embodiments the system includes a data processor which calculates a rate of change for one or more of the parameters.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system according to embodiments of the invention involves performing or completing selected tasks or steps manually, automatically, or a combination thereof. Moreover, according to actual instrumentation and equipment of exemplary embodiments of methods, apparatus and systems of the invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1 is a simplified flow diagram of a method according to some exemplary embodiments of the invention;

FIG. 2 is a schematic representation of an article of furniture according to some exemplary embodiments of the invention; and

FIG. 3 is a schematic representation of a system according to some exemplary embodiments of the invention.

### DETAILED DESCRIPTION OF THE EMBODIMENT

Aspects of the present disclosure relate to systems and methods and/or articles of furniture for emotion detection.

Specifically, some embodiments of the invention are used to evaluate content and/or to predict undesirable behavior.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It is expected that during the life of this patent many non-contacting physiologic sensor types will be developed and the scope of the invention is intended to include all such new technologies a priori.

As required, detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

As appropriate, in various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally or alternatively, the data processor may access a non-volatile storage, for example, a magnetic hard disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials described herein for illustrative purposes only. The materials, methods, and examples not intended to be necessarily limiting. Accordingly, various embodiments may omit, substitute, or add various procedures or components as appropriate. For instance, the methods may be performed in an order different from described, and that various steps may be added, omitted or combined. In addition, aspects and components described with respect to certain embodiments may be combined in various other embodiments.

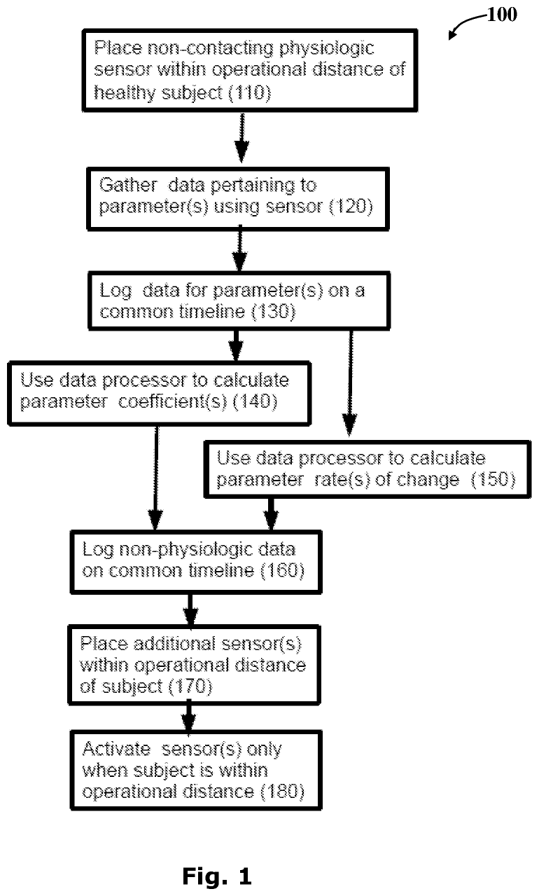

FIG. 1 is a simplified flow diagram of a method according to some examples of the invention indicated generally as 100.

Depicted exemplary method 100 includes placing 110 a non-contacting physiologic sensing apparatus within operational distance of a subject, for example outside of a hospital or medical clinic and gathering 120 data pertaining to at least one parameter selected from the group consisting of heart rate (HR), heart rate variability (HRV), respiration rate variability (RR), Cardiac Output calculation (CO) and, where appropriate, respiration volume (RV) using the sensing apparatus. The method may further include inferring the emotional state of the subject.

It is noted that, where the subject has a cardiovascular disease, monitored physiological parameters may not accurately correlate with emotional status. Accordingly, preferable implementation of the system may be limited only to subjects presenting no signs of cardiovascular problems.

In the depicted example, method 100 includes logging 130 data for each of the at least one parameter on a common timeline.

Alternatively or additionally, in some embodiments method 100 includes using 140 a data processor to calculate a coefficient based on two or more of the parameters. (example coefficients include, but are not limited to, HR/RR and HR/RV. In some embodiments the coefficient(s) are also plotted on the common timeline.

Alternatively or additionally, in some examples, method 100 includes using 150 a data processor to calculate a rate of change for one or more of said parameters. (e.g. ΔHR or ΔRR or ΔRV)

In some examples of method 100 the sensor is positioned in or on or near a seat. In some examples the sensor is embedded inside the seat (as part of the manufacturing process). In other implementations, the sensor is provided as an independent unit reversibly attachable to the seat. Optionally the user may remove the sensor if they wish to not be monitored.

In the depicted example, method 100 includes logging 160 non-physiologic data on the common timeline. In some examples, the non-physiologic data is content presented to the healthy subject (see 110).

In some examples, data clustering based on signals representing temporal blocks (e.g. 10 seconds) of physiologic parameters signals is performed and an algorithm is applied to each temporal block. In some examples use of temporal blocks contributes to an increase in machine learning capability. In some examples machine learning uses a set of data as an input, compares the input to previous measurements and makes a prediction, based on a database of previously acquired measurements using a statistical analysis.

In some examples method 100 includes placing 170 one or more additional non-contacting physiologic sensors within operational distance of the healthy subject. Where appropriate, data from these additional sensors is routed via the first sensor or separately. In some examples, method 100 does not employ a camera and/or a microphone as a sensor.

Alternatively or additionally, the method 100 may include activating 180 the sensor(s) only when the subject is within the operational distance.

### Articles of Furniture

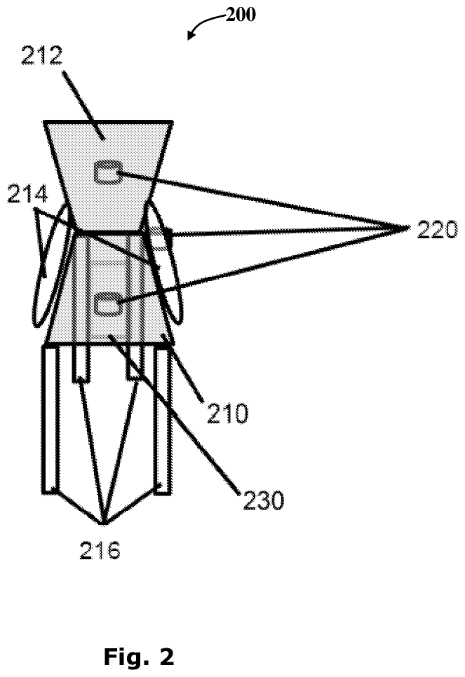

FIG. 2 is a schematic representation of an article of furniture for supporting a non-recumbent subject according to some implementations of the invention indicated generally as 200.

The term ‘non-recumbent’ is used to refer to a subject supported in a position that is not prone, supine or prostrate. For example a seated subject would be non-recumbent, even if that seat was reclining, however a subject lying upon a bed, by contrast, would be considered recumbent.

Depicted article of furniture 200 includes a seating platform 210 for supporting the non-recumbent subject and one or more non-contacting physiologic sensors 220 (three are depicted) installed in or on item of furniture 200. For purposes of this specification and the accompanying claims, the term “article of furniture” may exclude beds, because a bed has no seating platform. For purposes of this specification and the accompanying claims, “article of furniture” includes, but is not limited to, chairs, benches, car seats, airplane seats, train seats and movie theatre seats. In some examples, sensor 220 is installed in or below or on an edge of seat 210.

In some examples, article of furniture 200 includes a back 212 and sensor 220 is installed in or on back 212 (e.g. on a side of back 212 facing away from a person seated on seat 210).

In some examples, article of furniture 200 includes an armrest 214 and sensor 220 is installed in or on or below armrest 214.

The depicted article of furniture 200 includes two or more of sensors 220 installed at different locations.

Various non-contacting physiological sensors 220 may be incorporated into the system as suit requirements. For illustrative purposes only, by way of non-limiting example, one commercially available respiration sensor is the Xethru® X4M200, which tracks both respiration and movement. Another available sensor is the Sharp® DC6M4JN3000 microwave sensor module, which may provide non-contact detection of body motion, heartbeats and breathing. Yet another possible sensor available is muRata® SCA10H which is a heart and respiration monitor typically embedded into beds to monitor recumbent subjects but which, according to the current disclosure may be adapted and incorporated into systems for monitoring non-recumbent subjects.

Although three possible sensors are described above, in order to illustrate how the system may be implemented, it will be appreciated that other sensors may alternatively be used with the current system.

In still other examples of emotion monitoring systems, image capture sensors such as cameras, CCDs, IR detectors and the like may provide vision based solutions for assessment of behavioral and emotional reaction.

It has been found that a multi-modal approach may be used for monitoring emotional state by tracking psychophysiological states, and their corresponding effects upon a subjects body (e.g. facial expressions, tone of voice, body temperature, skin moisture level, heart and respiration and more).

By integrating a camera, along with a microphone, and various physiological monitors (i.e. ECG, EEG, GSR, etc.) in order to capture visual indications of the subject for a more comprehensive understanding of the subject's psychological change in correlation with a changing scenario and/or environment.

Surprisingly, we have found that such an approach produces more reliable and precise results than previous approaches, which were based only on using one tool for evaluating such response, for example, only a camera, or only a microphone, or only a physiological monitoring such as an ECG.

Accordingly, for example, by placing a driver facing camera, which records different head and body positions, inside the cabin of a car alertness and distraction may be detected and monitored. Similar devices may be used in other environments. This vision element will allow further assessment of the person under test, by evaluating not only physiological arousal level, but also comfort level.

The above solution can be utilized for additional detection of body, head, eye movement and positioning, in order to derive certain emotional states, such as fatigue, distraction, and comfort level. One method for extraction of said features can be derived from an algorithm noted as KLT by Shao et al (see “Simultaneous Monitoring of Ballistocardiogram and Photoplethysmogram Using a Camera” IEEE Trans Biomed Eng. 2017 May; 64(5):1003-1010 which is incorporated herein in its entirety by reference.)

In some examples, sensor 220 includes reversible attachment hardware. For purposes of this specification and the accompanying claims, the term “reversible attachment hardware” includes, but is not limited to, one or more magnets, hook and eye fasteners (e.g. VELCRO®), one or more screws, one or more nails, one or more rivets and one or more staples.

In some examples, article of furniture 200 includes an occupancy detector 230 that activates sensor 220 only when seat 210 is occupied. According to various exemplary embodiments of the invention, occupancy detector 230 detects one or more of weight, pressure, displacement and angle.

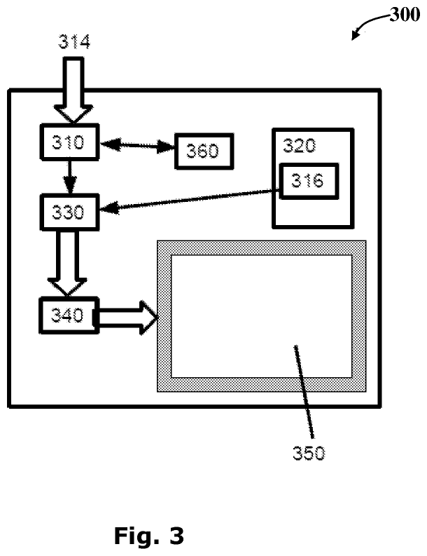

Reference is now made to the system of FIG. 3. FIG. 3 is a schematic representation of a system indicated generally as 300.

In some implementations, the system 300 includes a first memory buffer 310 receiving (and storing) a time stamped output 314 from a non-contacting physiologic sensor (e.g. 220 in FIG. 2) monitoring a subject exposed to time stamped content and a second memory buffer 320 containing the time stamped content 316.

In the depicted example, system 300 includes a temporal registration module 330 registering time stamped output 314 on time stamped content 316 to produce a log file 340 of physiologic responses to the content.

According to various examples temporal registration module 330 employs at least one clock selected from the group consisting of a clock embedded in content 316, a clock associated with the sensor (e.g. 220 in FIG. 2) and a clock providing an output signal to both the sensor and a content presentation device.

Alternatively or additionally, in some implementations the system 300 includes a presentation module 350 which presents time stamped output 314 from the non-contacting physiologic sensor concurrently and/or synchronously with content 316. In some embodiments temporal registration contributes to an ability to comprehend an emotional response of one or more viewers to specific items or sequences in the content.

In some examples the presentation module 350 presents time stamped output 314 from the non-contacting physiologic sensor graphically. Alternatively or additionally, the presentation module 350 may present time stamped output 314 from the non-contacting physiologic sensor numerically.

Alternatively or additionally, the system 300 may include a data processor 360 that calculates a coefficient based on two or more of said parameters. Examples of coefficients are HR/RR and HR/RV. Alternatively or additionally, in some embodiments system 300 includes a data processor 360 that calculates a rate of change for one or more of the physiologic parameters. (For example ΔHR or ΔRR or ΔRV)

### Possible Use Scenarios

In some examples, sensor 220 serves as a security measure by detecting early signs of aggression. In some embodiments these early signs of aggression are predictive of a violent or criminal incident. For example, a sensor installed in an airplane seat can be used to provide early warning for an impending hi-jacking.

Alternatively or additionally, in some implementations presentation of time stamped output 314 from the sensor (e.g. 220) concurrently and/or synchronously with content 316 on presentation module 350 contributes to an ability to comprehend an emotional response of one or more viewers to specific items or sequences in the content.

In some examples output 314 is averaged for a population of content viewers.

### Occupancy Detectors

In some implementations, an occupancy detector contributes to a reduction in battery and/or power consumption related problems. According to these embodiments, when a person is not seated a normally off sensor does not provide an “on” signal to the system. According to various exemplary embodiments of the invention employ pressure sensors and/or gyro and/or movement sensors and/or NFC (near field contact) for phone authentication.

One example of an occupancy detector suitable for use in this context is a Sensor force resistor from Interlink Electronics (P/N: 30-73258). A person of ordinary skill in the art will be able to incorporate other occupancy detectors into various embodiments of the invention using this specification as a guide.

### Communication Protocols

In some examples, sensor 220 (FIG. 2) produces an output 314 (FIG. 3). According to various implementations the invention output 314 is transmitted to memory buffer 310 via wired or wireless communication.

Alternatively or additionally, where appropriate, time stamped content 316 is transmitted to memory buffer 320 via wired or wireless communication.

Suitable wireless communication protocols for use in context of the various embodiments of the invention include, but are not limited to Bluetooth, Wi-Fi, infrared, RF and microwave.

Suitable wired communication protocols for use in context of the various embodiments of the invention include, but are not limited to USB, FlexRay protocol, SPI, JTAG, and CAN bus.

Alternatively or additionally, memory buffers 310 and/or 320 may be provided as, or receive data from, an external drive such as a flash drive.

### Selected Advantages of the System

Data acquisition without cameras and microphones contributes to an increase in anonymity for monitored subjects.

The principles and operation of a system and/or method and/or article of furniture according to certain examples may be better understood with reference to the drawings and accompanying descriptions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and do not limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various examples may exclude any specific feature, part, component, module, process or element which is not specifically disclosed herein.

Specifically, the invention has been described in the context of content evaluation but might also be used as a lie detector test or interrogation tool.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like intended to include all such new technologies a priori.

As used herein the term “about” refers to at least ±10%. The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6, should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the invention, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the invention has been described in conjunction with specific embodiments thereof, it is evident that other alternatives, modifications, variations and equivalents will be apparent to those skilled in the art. Accordingly, it is intended to embrace all such alternatives, modifications, variations and equivalents that fall within the spirit of the invention and the broad scope of the appended claims. Additionally, the various embodiments set forth hereinabove are described in terms of exemplary block diagrams, flow charts and other illustrations. As will be apparent to those of ordinary skill in the art, the illustrated embodiments and their various alternatives may be implemented without confinement to the illustrated examples. For example, a block diagram and the accompanying description should not be construed as mandating a particular architecture, layout or configuration.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a computer-readable medium such as a storage medium. Processors may perform the necessary tasks.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. The scope of the disclosed subject matter is defined by the appended claims and includes both combinations and sub combinations of the various features described hereinabove as well as variations and modifications thereof, which would occur to persons skilled in the art upon reading the foregoing description.

1. A method for predicting emotional state by monitoring a non-recumbent subject outside a medical institution, the method comprising:
(a) placing a non-contacting physiologic sensor within operational distance of the subject;
(b) gathering, using said non-contacting physiologic sensor, data pertaining to at least one parameter selected from the group consisting of heart rate, respiration and body-vibrations;
(c) determining from the data at least one physiological parameter selected from the group: heart rate variability, respiration rate variability, Cardiac Output calculation, respiration volume and combinations thereof; and
(d) inferring said emotional state of said subject.

2. The method according to claim 1, comprising:
logging on a common timeline at least one of: data for each of said at least one parameter, and said non-physiologic data on said common timeline.

3. The method according to claim 1, further comprising using a data processor to calculate at least one of:
a coefficient based on two or more of said parameters, and
a rate of change for one or more of said parameters.

4. The method according to claim 1, wherein said sensor is integrated into a seat.

5. The method according to claim 1, comprising placing one or more additional non-contacting physiologic sensors within operational distance of said healthy subject.

6. The method to claim 1, comprising activating said sensor only when said subject is within said operational distance.

7. An article of furniture for predicting emotional state of a subject, the article of furniture comprising comprising:
(a) a seating platform; and
(b) a non-contacting physiologic sensing apparatus operable to monitor a subject sitting in said seating platform and to gather data pertaining to at least one physiological parameter of said subject selected from a group consisting of: heart rate, respiration, body-vibrations, heart rate variability, respiration rate variability, cardiac output calculation, respiration volume and combinations thereof.

8. The article of furniture according to claim 7, wherein said at least one non-contacting physiologic sensing apparatus comprises at least one parameter sensor installed at a location selected from the group consisting of within said seating platform, below said seating platform, and on an edge of said seating platform.

9. The article of furniture according to claim 7, further comprising at least one additional feature selected from: a back support, an armrest, a seat belt, a head rest, a footrest and combinations thereof, and wherein said non-contacting physiologic sensing apparatus is incorporated into said additional feature.

10. The article of furniture according to claim 7, wherein said at least one non-contacting physiologic sensing apparatus comprises at least two parameter sensors installed at different locations on said furniture.

11. The article of furniture according to claim 7, wherein said at least one non-contacting physiologic sensing apparatus includes reversible attachment hardware.

12. The article of furniture according to claim 7, wherein said at least one non-contacting physiologic sensing apparatus further comprises an occupancy detector that activate a parameter sensor only when said seat is occupied.

13. The article of furniture of claim 7 for predicting emotional state of a subject, the article further comprising at least one of:
an image capture sensor configured and operable to record visual indications of the subject; and
a microphone configured and operable to record audio indications of the subject.

14. A system comprising:
(a) the article of furniture of claim 7;
(b) a first memory buffer receiving a time stamped output from a non-contacting physiologic sensor monitoring a subject exposed to time stamped content;
(c) a second memory buffer containing said time stamped content; and
(d) a temporal registration module registering said time stamped output on said time stamped content to produce a log file of physiologic responses to said content.

15. The system according to claim 14, wherein said a temporal registration module employs at least one clock selected from the group consisting of a clock embedded in said content, a clock associated with said sensor and a clock providing an output signal to both said sensor and a content presentation device.

16. The system according to claim 14, comprising a presentation module that presents said time stamped output from said non-contacting physiologic sensor concurrently with said content.

17. The system according to claim 14, wherein said presentation module presents said time stamped output from said non-contacting physiologic sensor graphically.

18. The system according to claim 14, wherein said presentation module presents said time stamped output from said non-contacting physiologic sensor numerically.

19. The system according to claim 14, comprising a data processor which calculates a coefficient based on two or more of said physiological parameters.

20. The system according to claim 14, comprising a data processor which calculates a rate of change for one or more of said physiological parameters.
